(19) United States
(12) Patent Application Publication
(10) Pub. No.: US 2005/0176110 A1
(43) Pub. Date: Aug. 11, 2005

(54) Process for chromatographic separation of nucleosides

(75) Inventors: Matti Leisola, Espoo (FI); Jouni Jokela, Vantaa (FI)

Correspondence Address: BIRCH STEWART KOLASCH & BIRCH, PO BOX 747, FALLS CHURCH, VA, 22040-0747, US

(73) Assignee: TEKNILLINEN KORKEAKOULU, TKK (FI)

(21) Appl. No.: 10/516,071

(22) PCT Filed: May 30, 2003
(86) PCT No.: PCT/FI03/00430

(30) Foreign Application Priority Data
May 29, 2002 (FI) 20021002

(51) Int. Cl. C07H 21/04; C12P 19/30

(52) U.S. Cl. 435/89; 536/25.4

(57) ABSTRACT

The invention relates to a process for chromatographic separation and purification of natural and synthetic nucleosides, deoxynucleosides and corresponding bases by using cross-linked xylose isomerase. In particular, nucleosides are separated from biological material, especially industrial side streams.

### FIELD OF THE INVENTION

This invention relates to protein technology and concerns particularly a new process for the separation and purification of nucleosides and nucleoside bases from biological material by using cross-linked xylose isomerase crystal stationary phase.

### BACKGROUND OF THE INVENTION

Biological material contains large amounts of valuable chemical compounds. Many food and biotech industry's waste and side streams contain numerous valuable bioorganic molecules in dilute concentrations. Amino acids and nucleosides are e.g. produced from animal waste or yeast biomasses. Quantitatively significant side streams come from corn, potato, grain and sugar manufacturing as well as from various microbial fermentation processes. Sugar beet molasses and pulp and paper industries side streams have become a rich source of a variety of chemicals including molecules like betaine, amino acids, L-arabinose, rhamnose, galactose, mannose and others. Composition of sugar beet molasses depends on the origin of the raw material and manufacturing process of sucrose but in general is well known (Schiweck, 1994). The main component of sugar beet molasses is sucrose but in addition it contains many other mono- and oligosaccharides and other compound groups like ribonucleosides, RNA-bases, amino acids, organic acids, polyols, vitamins and betaine. In terms of value in kilogram of sugar beet molasses amino acids as a compound group have the highest market value. Raffinose is the most valuable single compound in sugar beet molasses. Of the organic acids, in particular only γ-aminobutyric acid, pyrrolidonecarboxylic acid and lactic acid have commercial value. The market value of ribonucleosides is at the same level than market value of the earlier mentioned three organic acids together.

Chromatography is an industrially used technology in the separation of compounds like sucrose, betaine, inositol and amino acids from the sugar beet and sugar cane molasses (Paananen and Kuisma, 2000). Literature describes many analytical or small scale chromatographic methods for the separation of ribonucleosides. Cation-exchange chromatography was the originally applied method. The use of ion-exchange chromatography for the separation of nucleosides from sugar beet molasses was tested already in the 60's (Stark, 1962). Silica based reversed-phase chromatography introduced in the 70's has displaced all other chromatographic methods in analytical scale separation of nucleosides. In reversed-phase chromatography, volatile buffers, which facilitate the sample recovery in preparative chromatography, have shown to be applicable also for the separation of nucleosides (Ip et al., 1985). Reversed-phase flash chromatography has potential as a large scale method to separate nucleosides from their mixtures (O'Neill, 1991). Preparative scale separation of nucleosides by adsorption chromatography has also been studied (Aoyagi et al., 1982). Also the long known ability of neutral sugars to form negatively charged borate complexes having chromatographically different properties, can be used in nucleoside separations (Glad, 1983; Pal, 1978).

A fairly new way to use proteins is a cross-linked crystalline protein. In this form a protein becomes stable, insoluble in water and solvents, and mechanically rigid. Already in the 1960s crystallised proteins were stabilised by cross-linking for X-ray crystallographical studies. A common cross-linking agent is glutaraldehyde. Visuri (U.S. Pat. No. 5,437,993) prepared the first industrial cross-linked crystalline enzyme product from xylose isomerase (generally known as glucose isomerase). Cross-linked xylose isomerase is insoluble in water, wherefore it can be used in a chromatographic column without a separate carrier. Such a chromatographic column, filled merely with cross-linked crystalline protein matrix has new separation modes and efficiencies due to the pore structure of the crystals and the lack of inert carrier material.

Vilenchik et al., 1998, proved that different protein crystals can be used as chromatographic separation material. Particularly interesting was the fact that a protein crystal was capable of separating different enantiomers from each other. Altus Biologics Inc. has filed a patent application relating to the use of crystalline proteins as a universal separation material (PCT application WO 98/13119).

It has also earlier been shown that a column packed with cross-linked xylose isomerase crystals (CLXIC) separated different compound classes according to different mechanisms (Pastinen et al., 2000). As a porous material, CLXIC separated a molecular weight series of polyethylene glycols according to their size. A series of n-alcohols was separated according to their hydrophobicity. The mechanism behind amino acid separation was not so evident as there was no clear correlation between amino acid retention in the CLXIC-column and the 437 different physicochemical and biological properties of amino acids obtained from Amino Acid Index Database, (http://www.genome.ad.jp/dbget/aaindex. html; Shuichi et al., 1999). CLXIC-material was not a strong chiral phase in amino acid separation but showed its chiral separation potential against D/L-arabitol (Pastinen et al., 2000) showing also specific affinities towards other polyols, especially towards xylitol and sorbitol (Pastinen et al., 1998). Substrate spectrum of Streptomyces rubiginosus xylose isomerase is broad consisting of all D- and L-pentose sugars and many hexose sugars (Pastinen et al., 1999a,b). With pentose sugars, the CLXIC-column functioned as a chromatographic reactor concomitantly isomerizing and separating reactant sugars (Jokela et al., 2002). At least one hexose sugar, D-talose, had affinity against CLXIC-material without reaction (Jokela et al., 2002). All these chromatographic separation characteristics of the CLXIC-column together with its mechanical rigidity (Pastinen et al., 2000) and the extreme stability of the enzyme itself even in soluble form (Volkin and Klibanov, 1988) refer to the separation ability of the CLXIC-column towards nonphosphorylated ribonucleosides. These compounds are relatively small molecules able to penetrate into the pores of CLXI-crystals and contain D-ribose as a structural component. Ribonucleosides uridine, cytidine, adenosine and guanosine originating from natural sources are present in unbound form in different industrial process streams. Such a quantitatively important process stream is sugar beet molasses from sucrose refining.

### SUMMARY OF THE INVENTION

The invention relates to a new way to use cross-linked crystalline xylose isomerase for performing separation and purification of nucleosides. This enables manufacturing of higher value nucleosides from industrial process streams, including low value waste process streams, from which the separation of nucleosides could be otherwise uneconomical.

The invention concerns a process for the separation and purification in which a chromatographic column is packed with cross-linked crystalline xylose isomerase, whereafter a nucleosides (or nucleoside bases) containing solution, such as a sugar beet molasses or a fraction of it, is applied to the column, the column is eluted, and fractions containing pure or enriched nucleosides are collected from the out-let stream. The fractions containing the enriched nucleosides can be reapplied in the column until the nucleosides are in the desired purity.

As eluent, water and aqueous solutions are preferred.

The invention can be used for a wide variety of both natural and synthetic nucleosides. In connection with the present invention, the nucleosides include, but are not restricted, to nucleosides, deoxynucleosides and corresponding bases, such as uridine, cytidine, adenosine, guanosine, deoxyguanosine, thymidine, ribothymidine, xanthosine, inosine, hypoxantihine, deoxyadenosine, deoxycytidine, etc.

The invention concerns particularly a chromatographic process for the separation and purification of uridine, cytidine, adenosine, guanosine, deoxyguanosine and thymidine, the process being characterized in that cross-linked crystalline xylose isomerase is packed into a liquid chromatographic column, whereafter nucleoside containing solution, such as sugar beet molasses or fractions of it, is applied into the column which is eluted with water and at least partially separated nucleosides are collected from the effluent.

The invention also concerns the use of cross-linked xylose isomerase for the separation and/or purification of nucleosides.

According to the preferred embodiments of the process according to the invention
uridine is purified from sugar beet molasses or from an industrially purified fraction of it,
cytidine is purified from sugar beet molasses or from an industrially purified fraction of it,
adenosine is purified from sugar beet molasses or from an industrially purified fraction of it,
guanosine is purified from sugar beet molasses or from an industrially purified fraction of it,
deoxyguanosine is purified from sugar beet molasses or from an industrially purified fraction of it, and
thymidine is purified from sugar beet molasses or from an industrially purified fraction of it.

### BRIEF DESCRIPTION OF THE DRAWINGS

FIG. 1.

Retention of ribonucleosides in the CLXIC-column eluted with 1 ml H₂O/min at 50° C. Ethylene glycol (EG) is a small molecule (MW 62) not interacting with CLXIC-material and eluting near V₀. Small front peak in the chromatograms of adenosine and guanosine is from NaOH added to improve solubility.

FIG. 2.

Temperature dependence of the retention of certain sugar beet molasses' components in the CLXIC-column eluted by 1 ml/min with 2 mM MgSO₄. Abbreviations: uridine (Urd), cytidine (Cyd), adenosine (Ado), guanosine (Guo), thymidine (dThd), uracil (U), cytosine (C), adenine (A), guanine (G), thymine (T), xanthine (X), myo-inositol (Inos), γ-amino-butyric acid (GABA), betaine (bet), D- and L-pyroglutamic acid (PGA), ethylene glycol (EG).

FIG. 3.

Retention of individual nucleosides from their synthetic mixture in the CLXIC-column. 100 mg of an equimass mixture of five nucleosides Urd, Cyd, dThd, Ado and Guo was eluted with 1 ml H₂O/min at 50° C.

FIG. 4.

Retention of individual nucleosides in the CLXIC-column when 0.1 ml or 1 ml of sugar beet molasses was eluted with 1 ml H₂O/min at 30° C. Upper panel: The original RI-signal is multiplied 3.65× and horizontal segmented line shows the four pooled fractions for 2^{nd} and 3^{rd} separations. Lower panel: vertical bars show the retention peaks of betaine, inositol and sucrose. Dashed line shows the elution of inorganic ions (conductivity, presented without an y-axis). Horizontal bar above the x-axis shows the elution of SBM brownish color.

### DETAILED DESCRIPTION OF THE INVENTION

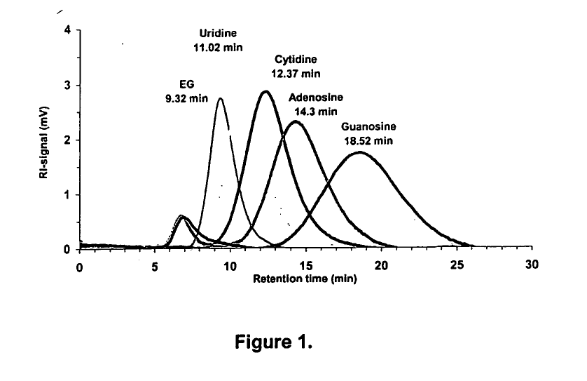

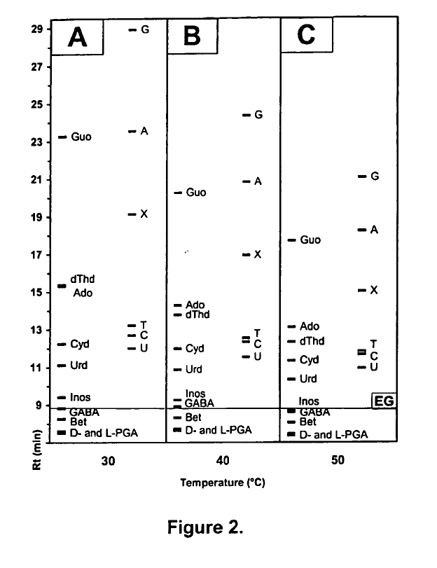

We have now observed that cross-linked crystalline xylose isomerase have some previously unknown specific interactions with nucleosides, deoxynucleosides and corresponding nucleoside bases. These observations have enabled utilization of column packed cross-linked crystalline xylose isomerase for the separation and purification of nucleosides from each other (FIG. 1). Nucleosides, deoxynucleosides and nucleoside bases interacting with cross-linked crystalline xylose isomerase are presented in FIG. 2.

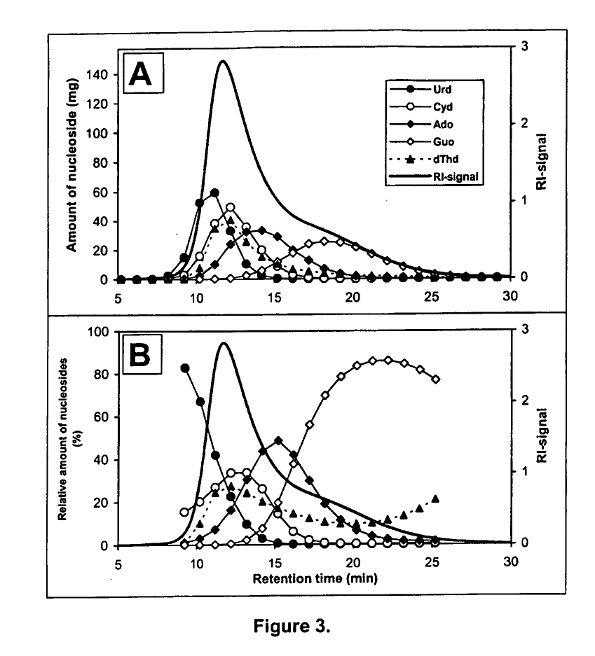

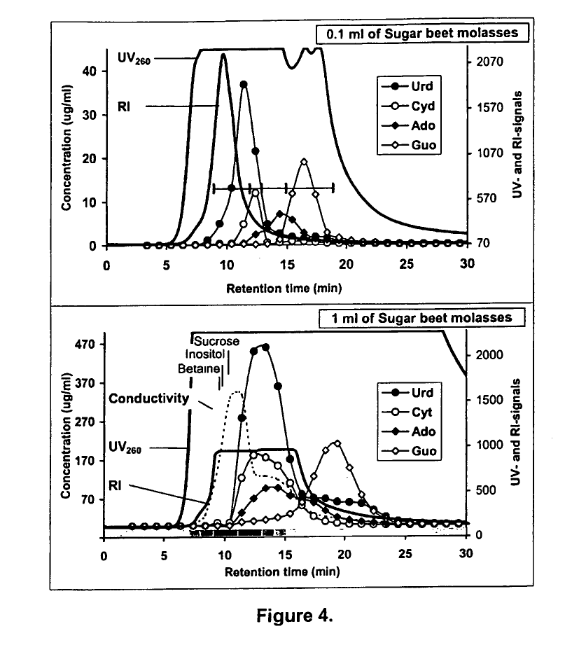

We have also observed that column packed cross-linked crystalline xylose isomerase can be used to separate and purify nucleosides from industrial streams, such as sugar beet molasses as efficiently as from artificial mixtures of nucleosides in water (FIGS. 3 and 4, upper panel).

Column packed cross-linked crystalline xylose isomerase is especially efficient in separation and purification of guanosine. Even when 10% of the liquid volume of the column is loaded with high viscous sugar beet molasses, guanosine separates from the other components as efficiently as from an artificial mixture of nucleosides in water (FIGS. 3 and 4, lower panel).

The temperature of the chromatographic column is preferably the is same as the temperature of the process stream to be purified. The temperature depends primarily on the cross-linked protein. The maximum temperature of a chromatographic column packed with cross-linked xylose isomerase is 70° C. When the temperature decreases, the nucleosides are eluted at greater distance from each other (FIG. 2). Separation has been made at a temperature of 5° C., where the nucleoside peaks were at the greatest distance from each other. The pH can vary within wide ranges, such as pH 4-10.

As raw material, any DNA and RNA containing biological material, such as biomass and biomass derivatives, including plant material, animal waste material and microbes, can be used. In particular, the invention is useful in the separation and/or purification of nucleosides from industrial side streams. In connection with the present invention, process streams from sugar manufacturing have been used by way of example.

The invention is described more closely by the following examples, which are merely intended to clarify the invention but not to restrict it.

### EXAMPLE 1

Separation of Uridine, Cytidine, Adenosine, Guanosine and Thymine from Their Water Solution

Cross-linked xylose isomerase crystals according to Visuri's U.S. Pat. No. 5,437,993 with mean diameter of 83 μm were prepared. The enzyme was packed in water slurry into a steel column, which was 300 mm in length and 7.8 mm in diameter. 100 mg of an equimass mixture of five nucleosides, uridine, cytidine, thymidine, adenosine and guanosine, in a water solution of 1 ml was applied to the column, the temperature of which was 50° C. The elution was carried out with 1 ml water/min. The elution of nucleosides from the column took 30 min (FIG. 3). The maximal concentrations of the nucleosides were 11.2 min for uridine, 12.2 min for cytidine and thymidine, 14.2 min for adenosine and 18.2 for guanosine. More than 50% pure uridine and guanosine could be collected at the beginning and at the end of the elution profile, respectively (FIG. 3B). Nucleosides collected were analyzed by HPLC using 150×4.6 mm Nova-Pak C18 (4 μm) column (Waters) or 100×3.9 mm XTerra RP₁₈ (3.5 μm) column (Waters) which was eluted at 1 ml/min or 0.5 ml/min, respectively, with 4 mM K-phosphate buffer (pH 5.8), containing 1% MeOH or without MeOH.

### EXAMPLE 2

Separation of Uridine, Cytidine, Adenosine and Guanosine from Sugar Beet Molasses

100 μl of the sugar beet molasses containing 470 g sucrose/l and 3.8 g of the four nucleosides together per liter was applied to the chromatographic column described in Example 1. The temperature of the column was 30° C. and the flow rate was 1 ml water/min. The elution of nucleosides from the column took 20 min (FIG. 4, upper panel). The maximal concentrations of the nucleosides were 11.4 min for uridine, 12.4 min for cytidine, 14.4 min for adenosine and 16.4 for guanosine. The %-ratios of uridine, cytidine, adenosine and guanosine in the nonfractionated sugar beet molasses and in the purified nucleoside solutions after 1^{st} purification cycle are presented in Table 1. Nucleosides collected were analyzed as described in Example 1.

### EXAMPLE 3

High Capacity Separation of Uridine, Cytidine, Adenosine and Guanosine from High Viscous Sugar Beet Molasses

1 ml of sugar beet molasses containing 620 g sucrose/l and 5.1 g of the four nucleosides together per liter was applied to a chromatographic column described in Example 1. The temperature of the column was 30° C. and the flow rate was 1 ml water/min. The elution of nucleosides from the column took 25 min (FIG. 4, lower panel). The maximal concentrations of the nucleosides were 13.4 min for uridine, 12.4 min for cytidine, 14.4 min for adenosine and 19.4 for guanosine. Nucleosides collected were analyzed as described in Example 1.

### EXAMPLE 4

Further Purification of Partially Separated Uridine, Cytidine, Adenosine and Guanosine

Fractions from the nucleoside separation carried out in Example 2. were further purified by pooling the uridine, cytidine, adenosine and guanosine fractions as shown in FIG. 4, upper panel in which horizontal segmented line shows the location of the pools. Vacuum concentrated solutions (volume <100 μl) were re-fractionated by CLXIC-column in a temperature of 30° C. and with a flow rate of 1 ml water/min. Uridine, cytidine, adenosine and guanosine were again collected into four separate fractions. The %-ratios of uridine, cytidine, adenosine and guanosine in the nonfractionated sugar beet molasses and in the purified nucleoside solutions after 1^{st} and 2^{nd} purification cycle is presented in Table 1. Nucleosides collected were analyzed as described in Example 1.

TABLE 1
Percentage ratios of nucleosides in crude sugar beet molasses
and in the four nucleoside solutions obtained from two
enrichment cycles of 100 μl of the sugar beet molasses
containing 470 g/l of sucrose. Adenosine from the 2nd
enrichment cycle was not detected for unknown reason.

| | % of nucleoside from total | | | |
|---|---|---|---|---|
| | amount of nucleoside | | | |
| | | | Adeno- | Guano- |
| | Uridine | Cytidine | sine | sine |
| Crude sugar beet molasses | 40 | 20 | 15 | 25 |
| 1^{st} pool of enriched uridine | 89 | 9 | 1 | 1 |
| 2^{nd} pool of enriched uridine | 94 | 6 | 0 | 0 |
| Crude sugar beet molasses | 40 | 20 | 15 | 25 |
| 1^{st} pool of enriched cytidine | 60 | 33 | 7 | 0 |
| 2^{nd} pool of enriched cytidine | 35 | 57 | 7 | 1 |
| Crude sugar beet molasses | 40 | 20 | 15 | 25 |
| 1^{st} pool of enriched adenosine | 36 | 5 | 52 | 7 |
| 2^{nd} pool of enriched adenosine | | | ND | |
| Crude sugar beet molasses | 40 | 20 | 15 | 25 |
| 1^{st} pool of enriched guanosine | 10 | 4 | 17 | 69 |
| 2^{nd} pool of enriched guanosine | 1 | 2 | 2 | 96 |

ND = not detected.

Literature

Aoyagi S, Hirayanagi K, Yoshimura T, Ishikawa T. 1982. Preparative separation of nucleosides and nucleotides on a non-ionic gel column. J Cromatogr 253:133-137.

Altus Biologics Inc. 1997. PCT application WO 98/13119.

Glad M J, Ohison S A, Hansson L H, Mansson M O, Larsson P O, Mosbach K H. 1983. Separation agent. U.S. Pat. No. 4,406,792.

Ip C Y, Ha D, Morris P W, Puttemans M L, Venton D L. 1985. Separation of nucleosides and nucleotides by reversed-phase high performance liquid chromatography with volatile buffers allowing sample recovery. Anal Biochem 147:180-185.

Jokela J, Pastinen O, Leisola M. 2002. Isomerization of pentose and hexose sugars by an enzyme reactor packed with cross-linked xylose isomerase crystals. Enzyme Microb Tech, in press.

O'Neill I A. 1991. Reverse phase flash chromatography: a convenient method for the large scale separation of polar compounds. Synlett 9:661-662.

Paananen H and Kuisma J. 2000. Chromatographic separation of molasses components. Zuckerindustrie 125:978-981.

Pal B C. 1978. Novel application of sugar-borate complexation for separation of ribo-, 2′-deoxyribo-, and arabinonucleosides on cation-exchange resin. J Chromatogr 148:545-548.

Pastinen O, Visuri K, Leisola M. 1998. Xylitol purification by cross-linked glucose isomerase crystals. Biotech Techniques 12:557-560.

Pastinen O, Visuri K, Schoemaker H E, Leisola M. 1999a. Novel reactions of xylose isomerase from Streptomyces rubiginosus. Enzyme and Microb Tech 25:695-700.

Pastinen O, Schoemaker H E, Leisola M. 1999b. Xylose isomerase catalyzed novel hexose epimerization. Biocatal Biotrans 17:393-400.

Pastinen O, Jokela J, Eerikainen T, Schwabe T, Leisola M. 2000. Cross-linked glucose isomerase crystals as a liquid chromatographic separation material. Enzyme Microb Tech 26:550-558.

Schiweck H. 1994. Zusammensetzung von Zuckerbenmelassen. Zuckerindustrie 119:272-282.

Shuichi K, Ogata H, Kanehisa M. 1999. AAindex: amino acid index database. Nucleic Acids Res 27:368-369.

Stark J B. 1962. Use of ion-exchange resins to classify plant nitrogenous compounds in beet molasses. Anal Biochem 4:103-109.

Vilenchik L Z, Griffith J P, St Clair N, Navia M A, Margolin A L. Protein crystals as novel microporous materials. J Am Chem Soc 120: 4290-4294.

Visuri K. 1995. Preparation of cross-linked glucose isomerase crystals. U.S. Pat. No. 5,437,993; (Application date 1989).

Volkin D B, Klibanov A M. 1988. Mechanism of thermoinactivation of immobilized glucose isomerase. Biotechnol Bioeng 33:1104-1111.

1. A process for the separation and purification of nucleosides, characterized in that cross-linked crystalline xylose isomerase is packed into a chromatographic column, whereafter nucleosides containing solution is applied to the column, eluted and the fractions containing nucleosides are collected from the effluent.

2. The process according to claim 1, characterized in that fractions containing pure nucleosides are collected from the effluent.

3. The process according to claim 1, characterized in that fractions enriched with nucleosides are collected from the effluent.

4. The process according to claim 1 or 3, characterized in that the fractions containing the enriched nucleosides are reapplied in the column until the nucleosides are in the desired purity.

5. The process according to claim 1, characterized in that uridine, cytidine, adenosine, guanosine, deoxyguanosine and/or thymidine are separated and collected from the effluent.

6. The process according to claim 1, characterized in that the elution is made with weater or an aqueous solution.

7. The process according to claim 1, characterized in that the industrial process stream origins from natural raw materials containing nucleosides.

8. The process according to claim 7, characterized in that the industrial process stream is from sugar manufacturing.

9. The process according to claim 8, characterized in that the industrial process stream from sugar manufacturing is partially purified.

10. The process according to claim 1, characterized in that the industrial process stream is sugar beet molasses.

11. The process according to claim 1, characterized in that the industrial process stream is sugar beet molasses, from which other valuable compounds, such as betaine, inositol, raffinose, have already been separated.

12. Use of cross-linked xylose-isomerase for the separation of nucleosides.
